(19) United States
(12) Patent Application Publication
(10) Pub. No.: US 2017/0274170 A1
(43) Pub. Date: Sep. 28, 2017

(54) METHOD AND GAS ANALYSIS UNIT FOR DETERMINING A CHANCE TO ENABLE A ZEROING OF GAS ANALYSIS

(71) Applicant: General Electric Company, Schenectady, NY (US)

(72) Inventors: Heikki Haveri, Huhmari (FI); Timo Merilainen, Klaukkala (FI)

(73) Assignee: General Electric Company, Schenectady, NY (US)

(21) Appl. No.: 15/592,698

(22) Filed: May 11, 2017

Related U.S. Application Data
(62) Division of application No. 14/191,646, filed on Feb. 27, 2014.

(51) Int. Cl. A61M 16/08; A61M 16/00; G01J 5/02; G01J 5/10; G01N 21/27; G01N 21/31; G01N 21/3504; G01N 33/497; A61M 16/01; A61M 16/04; G01N 21/85

(52) U.S. Cl. CPC A61M 16/085; A61M 16/0051; G01N 21/274; G01N 21/314; G01N 21/3504; G01J 5/027; G01J 5/10; G01N 33/497; A61M 16/0833; A61M 2205/3313; A61M 2205/3368; A61M 2230/435; A61M 2230/437; A61M 16/01; G01J 2005/106; A61M 2230/432; A61M 16/04; G01N 2021/8578

(57) ABSTRACT

A method for determining a chance to enable a zeroing of gas analysis is disclosed herein. The method includes emitting radiation, and receiving emitted radiation, the received radiation comprising a first wavelength range absorbed by the at least one desired gas component and one or more disturbing factor, and a second wavelength range absorbed by the disturbing factor, the first wavelength range differing from the second wavelength range. The method also includes providing to a processing unit a first signal data indicative of a concentration of the at least one desired gas component and absorption of the disturbing factor, and a second signal data indicative of absorption of the disturbing factor. The method also includes determining a stability of the first and second signal data as a function of time, and if they are substantially stable enabling the zeroing to improve a measurement accuracy.

### BACKGROUND OF THE INVENTION

This disclosure relates generally to a method and gas analysis unit for determining a chance to enable a zeroing of gas analysis.

During anesthesia or in critical care, patients are often mechanically ventilated instead of breathing spontaneously. The patient is connected to a patient circuit of a ventilator or an anesthesia machine by intubation or non-intubation. In intubation air endotracheal tube is inserted into a trachea so that a gas can flow through it into and out of a lung. In intubation the breathing circuit typically comprises the endotracheal tube, a Y-piece where inspiratory and expiratory tubes from the ventilator come together, as well as the ventilator. In non-intubation the endotracheal tube is replaced with a breathing mask, which covers nasal and or oral cavities so that a gas can flow through it into and out of a lung.

A mainstream gas analyzer normally comprises an airway adapter, which connects to a breathing gas measuring sensor. The airway adapter is placed into the breathing circuit between the endotracheal tube or the breathing mask and the breathing circuit Y-piece to allow the breathing gas to flow through the airway adapter to be measured. Usually the airway adapter is replaced between the patients, to prevent cross contamination between the patients, or less than every fourth day, normally at least every day, to prevent bacteria to grow over unacceptable levels increasing infection risk.

A common way to measure the gas concentration of some gases, especially carbon dioxide and commonly used volatile anesthetics, is based on the gas absorption of non-dispersive infrared radiation (IR) at measured gas specific wave lengths. A common problem in IR gas analyzers, especially in mainstream gas analyzers is the measurement signal drift or change during or between the measurements. The infrared radiation generated by the IR source traverses through the optical path comprising different boundary surfaces such as optical windows of a gas sampling cell of the airway adapter before reaching the measuring detector(s). The optical path of a gas sampling cell becomes replaced when the airway adapter is changed. There are optical differences between the gas sampling cells due to manufacturing tolerances, material differences and optical path deformation along time, which cause gas analyzer measurement signal differences or error between the airway adapters. The measurement signal error can include offset error that can be seen as inspired/expired gas concentration signal offset or gain error that can be seen as an increasing error proportional to the measured gas concentration.

It is a challenge in existing mainstream gas analyzers to take into account the optical path differences between the airway adapters. For example the changes in material and the thickness of optical windows cause changes in spectral transmission and changes in the diameter of optical window openings cause changes in transmission, which can be seen as offset error. The manufacturing tolerances of optical path length cause differences in gas absorption that can be seen as gain error. In addition the components such as IR-source and the measuring detectors may drift at the same time increasing all these errors.

In existing mainstream gas analyzers it is common to zero the offset type measurement signal errors manually. This means that always when the airway adapter is replaced into the sensor the measurement needs to be at least zeroed, but sometimes also calibrated. The zeroing is done manually from the host device, for example by choosing a correct selection from the host's software menus to start zeroing at the certain state of the manual routine. It is also common, depending on the technology, to use a separate zeroing cell that replaces the airway adapter in the sensor for the time of zeroing when the measurement signal offset error is removed. This needs an accurately defined work routine including manual work of placing the zeroing cell into the sensor for the time of zeroing and starting the zeroing from the host's software menus at correct time. In many cases the measurement signal gain error needs to be calibrated as well. This is commonly done by placing a special calibration cell, including calibration gas or properties comparable to calibrated gas, into the sensor for the time the calibration is performed. The calibration routine needs manual work of placing the calibration cell into the sensor for the time of calibration, but also for example choosing a correct selection from the host's software menus to calibrate the measurement.

It is important that the ventilation meets the patient's needs for a correct exchange and administration of oxygen (O₂), carbon dioxide (CO₂), nitrous oxide (N₂O), anesthetic agents and other gases. Anesthesiologists, respiratory therapists and other qualified clinicians use their professional skills to set ventilation parameters to optimally meet needs of the patient. The ventilation is often monitored with a respiratory monitor by measuring typically in real time concentrations of oxygen, carbon dioxide, nitrous oxide and anesthetic agent in a breathing gas.

The gas analyzer's manual and accurately defined zeroing and calibration routines generate additional work for the hospital personnel and reduce the time for taking care of patients. Zeroing and calibration routines are complex and time consuming especially as the care situations they are used are often demanding and cause risk for zeroing and calibration errors. If the zeroing or calibration routines are disregarded the sensor most probably show wrong values generating a risk for a patient.

### BRIEF DESCRIPTION OF THE INVENTION

The above-mentioned shortcomings, disadvantages and problems are addressed herein which will be understood by reading and understanding the following specification.

In an embodiment, a method for determining a chance to enable a zeroing of gas analysis, the gas analysis unit being configured outside the zeroing to obtain a signal indicative of at least one desired gas component of a respiratory gas for an analysis, the method includes emitting radiation by means of at least one radiation source and receiving emitted radiation in at least one radiation sensing detector. The received radiation includes a first wavelength range configured to be absorbed by the at least one desired gas component of the respiratory gas in case such gas component is present to the extent exceeding a reference gas concentration, which first wavelength range also being absorbed by one or more disturbing factor, in case such factor is present, and a second wavelength range configured to be absorbed by the disturbing factor, the first wavelength range differing from the second wavelength range. The method also includes providing by means of the at least one radiation sensing detector to a processing unit a first signal data indicative of a concentration of the at least one desired gas component and indicative of absorption of the disturbing factor, and a second signal data indicative of absorption of the disturbing factor. The method also includes determining in the processing unit a stability of the first and second signal data as a function of time, and if they are substantially stable enabling the zeroing to improve a measurement accuracy.

In another embodiment, a gas analysis unit for determining a chance to enable a zeroing of gas analysis, the gas analysis unit being configured outside the zeroing to obtain a signal indicative of at least one desired gas component of a respiratory gas for an analysis, the gas analysis unit includes a sensor having at least one radiation source configured to emit radiation, and at least one radiation sensing detector configured to receive emitted radiation. The received radiation includes a first wavelength range configured to be absorbed by the at least one desired gas component of the respiratory gas, in case such gas component is present to the extent exceeding a reference gas concentration, which first wavelength range also being absorbed by one or more disturbing factor, in case such factor is present, and a second wavelength range configured to be absorbed by the disturbing factor, the first wavelength range differing from the second wavelength range. The at least one radiation sensing detector is configured to provide a first signal data indicative of a concentration of the at least one desired gas component and indicative, of the disturbing factor, and a second signal data indicative of the disturbing factor. The gas analysis unit also includes a processing unit configured to receive the first and second signal data from the at least one radiation sensing detector, and configured to determine a stability of the first and second signal data as a function of time, and if they are substantially stable the processing unit is configured to enable the zeroing to improve a measurement accuracy.

Various other features, objects, and advantages of the invention will be made apparent to those skilled in art from the accompanying drawings and detailed description thereof.

### BRIEF DESCRIPTION OF THE DRAWINGS

FIG. 1 is a schematic perspective view of a gas analyzing unit in accordance with an embodiment in an operating environment;

FIG. 2 is a flow diagram illustrating a method for determining a chance to enable a zeroing of a gas analysis unit of FIG. 1 in accordance with an embodiment;

FIG. 3 is a flow diagram illustrating a method for determining a chance to enable a zeroing of a gas analysis unit of FIG. 1 in accordance with an another embodiment; and

FIG. 4 is a block diagram illustrating a partial detail of the methods in FIGS. 2 and 3.

### DETAILED DESCRIPTION OF THE INVENTION

Specific embodiments are explained in the following detailed description making a reference to accompanying drawings. These detailed embodiments can naturally be modified and should not limit the scope of the invention as set forth in the claims.

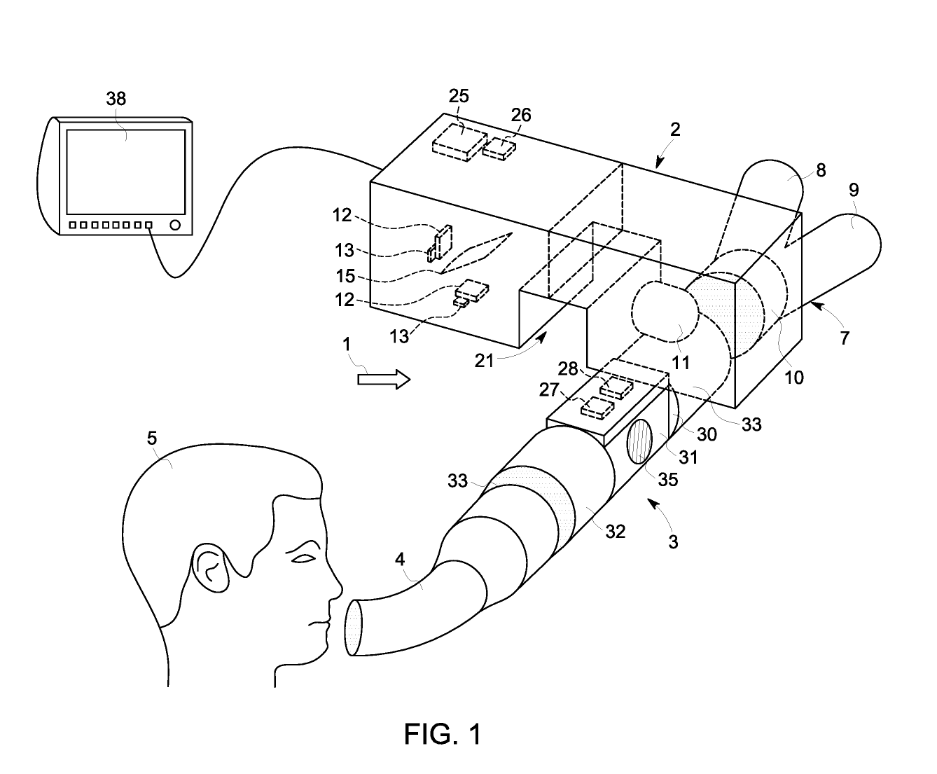

FIG. 1 shows a gas analyzing unit 1 comprising a sensor 2 such as a main stream sensor in a typical operating environment for making one of a qualitative or quantitative gas analysis of at least one respiratory gas like CO₂. The gas analyzing unit may possibly also comprise an airway adapter 3 connected through an endotracheal tube 4 to airways of a subject 5. The gas analyzing unit 1, especially the airway adapter 3, may also be connected to a branching unit 7 such as a Y-piece having at least three limbs, one of them being an inhalation limb 8 for inspired gas, a second one being an expiration limb 9 for expired gas, a third one being a combined inspiration and expiration limb 10 for both inspired and expired gases. The inhalation limb 8 is connectable to the inspiration tube (not shown) and the expiration limb 9 is connectable to the expiration tube (not shown), which both tubes may be connected to a ventilator (not shown) for allowing the gas exchange with the airways of the subject 5. The combined inspiration and expiration limb 10 is typically connected to the airway adapter 3.

The sensor 2 and airway adapter 3 connectable between the endotracheal tube 4 and the branching unit 7 as shown in FIG. 1 is especially designed for a mainstream measurement in which case a gas measurement is made directly in the main gas flow of the subject 5 and thus this can be done without taking samples out of this main gas flow. The airway adapter 3 includes a sampling chamber 31 between a connector 32 and a connector 33. The connector 32, which can be a female type, and the connector 33, which can be a male type, are used to connect the airway adapter 3 as well as the sensor 2 between the endotracheal tube 4 and the branching unit 7. The shape of the airway adapter 3 is not limited to any special shape or form, like described and shown in FIG. 1, but may be e.g. a breathing mask or a prong to guide breathing gas between the ventilator and the patient's oral or nasal cavities. Similarly the sampling chamber 31 is not limited to any special form but may be e.g. a tube or a part of tube or it may be a conventional special space with a rectangular cross sectional shape in to the direction of the gas flow. One or more optical windows 35, used for the gas concentration or component measurement based on for example absorption of an infrared radiation by different gases, are conventionally located on one side or both sides of the airway adapter 3. This kind of construction enables measuring the content of at least carbon dioxide, or other gases as well, in the breathing gas mixture.

When the sensor 2 comprising a radiation source 11, such as an infrared emitter, for emitting an infrared radiation through the sampling chamber 31 and at least one detector 12 for receiving the infrared radiation at least at two measurement wavelength ranges and creating an infrared absorption signal is used for measuring the gas concentration or gas composition of the subject, the airway adapter 3 is placed into a coupling point 21 such as a cavity of the sensor 2 so that breathing gases flowing through the endotracheal tube 4 and the branching unit 7 and through the sampling chamber 31 in airway adapter 3 can be analyzed by the sensor 2. The airway adapter 3 may be placed into the coupling point 21 of the sensor 2 preferably detachably, but naturally the airway adapter and the sensor can be integrated, too.

An electrical signal from the at least one detector 12 proportional to the measured gas concentration in the gas mixture of the breathing gas is transferred for further processing in to the processing unit 25 of the sensor 2 which may comprise an amplifier (not shown in FIG. 1), an analog to digital converter (not shown in FIG. 1), a memory 26 etc. to get a calculated concentration of the measured gas or to get an identification of a gas component of the respiratory gas flow. These analyzing results including this calculated concentration information and/or the analyzed gas component information, if needed, further be received by a host device 38 such as a monitor e.g. for further processing or only for displaying the information. Alternatively the electrical signal proportional to the measured gas concentration in the gas mixture of the breathing gas may be transferred from the sensor 2 to the host device 38 for further processing to get a calculated concentration of the measured gas. Also this is same with the gas composition or identification signal.

Although the processing of the signal is typically made in the sensor, it may also be made in the processing unit 27 of the airway adapter 3 comprising the amplifier (not shown in the Figure), the analog to digital converter (not shown in the Figure), the memory 28, the radio frequency transceiver (not shown in the Figure) etc. and a small battery (not shown in the Figure) that produces an electrical energy for the operation. In that case the processing unit with all these components may be around the airway adapter 3. As the airway adapter 3 and the sensor 2 integrated into one piece are preferably disposable rather than reusable and as the aim is advantageously to have very small sized combination of the sensor 2 and the airway adapter 3 as well as the low overall cost it may be more convenient to do further signal processing elsewhere.

The embodiment of the sensor 2 shown in FIG. 1 may comprise at least one, but advantageously two radiation sensing detectors 12 with different wavelength ranges. The first radiation sensing detector for the first wavelength range sensitive to measured gas and the second radiation sensing detector for the second wavelength range, which is not sensitive to measured gas. Actually the first wavelength range may be absorbed by at least one desired gas component of the respiratory gas in case such gas component is present to the extent exceeding a reference gas, such as normal atmospheric gas, concentration. The first wavelength range may also be absorbed by one or more disturbing factor, in case such factor is present. The second wavelength range may be absorbed by the disturbing factor, or typically substantially only by the disturbing factor. Further both the first and second wavelength ranges comprise a number of successive wavelengths, each wavelength range having a central wavelength. The central wavelength of the first wavelength range typically differs from the central wavelength of the second wavelength range or in other words the first and second wavelength ranges typically differ from each other. The collimated radiation beams are split with an optical beam slitter 15 to both radiation sensing detectors 12 to sense all changes due to absorption usually equally, except the second radiation sensing detector, which does not sense the measured gas as explained hereinbefore. Instead of two radiation sensing detectors there can be only one detector able to detect both the first and second wavelength ranges.

The gas analyzing unit 1 or the sensor 2 may also comprise at least one temperature sensing element 13, such as a thermistor, to measure the core temperature of the at least one radiation sensing detector 12. In case there are more than one radiation sensing detector, which are not in good thermal connection with each other, the number of the temperature sensing elements advantageously is same as the number of the radiation sensing element. The temperature sensing element 13 can provide to the processing unit a temperature signal data of the radiation sensing detector which signal is used to remove the signal error from the at least one radiation sensing detector caused by thermal changes due to such as the ambient temperature change, user touch or the temperature change of a flowing gas. The gas concentration signal is described by the signal difference between the two similar measurement wavelength ranges from the at least one radiation sensing detector 12 that may be corrected with the signals from the at least one temperature sensing element 13.

As explained hereinbefore it is also possible to have only one radiation sensing detector that measures at least two wavelength ranges in turns as the at least two optical windows pass the optical path filtering the gas specific wavelength ranges for the radiation sensing detector to be measured, such existing technology known as a spinning wheel or chopper wheel technology.

The emission of the infrared radiation source, the measurement signals of radiation sensing detectors or the whole optical system of the gas analyzer may change suddenly or drift in time causing gas concentration or detection signals to change or drift. The sudden change may be caused by for example the optical system including the sampling chamber, which may contaminate due to secretions or liquids in the breathing circuit, but also for example due to impurities such as grease from the user's hands, but also other parts such as optical cavities, which may oxidize, contaminate or change their shape due to mechanical elongation caused by ambient changes or mechanical shocks. The drift in time may be caused by for example the change in emission or the spectrum of emitted radiation of radiation source that may change due to oxidation of emitter filament, filling gas leakage, optical deteriorating or any mechanical change caused by the ambient changes or mechanical shocks. The measurement signals of radiation sensing detectors may change due to similar reasons as of the radiation source emitting for example infrared radiation.

The gas analyzing based on one wavelength range is sensitive to all kind of absorption. The optical windows 35 of the sampling chamber 31 absorb infrared radiation, which absorption is dependent on for example the thickness of the optical windows, the diameter of optical window's openings and the optical window's material properties, which cause differences between the airway adapters 3. There are differences between the sensors 2 as well, such as the optical path differences which depend on the diameters and surface finishing of optical path cavities, the optical window differences as with the airway adapters and differences between infrared radiation sources and receiving detectors which all cause absorption differences between the gas analyzers. Many of these differences cannot be controlled due to manufacturing tolerances and are difficult to eliminate without zeroing manually, frequently, after any change.

The gas analyzing based on the differential measurement at two wavelength ranges, the first wavelength range sensitive to wavelength range absorbed by the measured gas and the second wavelength range sensitive to wavelength range excluding the measured gas, is not sensitive to common mode absorption differences, such as described with one wavelength range measurement. However, the measurement based on the first and second wavelength ranges is sensitive to spectral absorption differences, if the absorption spectrum remains constant. In practice the optical windows 35 of the sampling chamber 31, located in the airway adapter 3, absorb infrared radiation differently depending on the measured wavelength. Thus the amount of infrared radiation the radiation source 11 emits and the at least one radiation sensing detector 12 receive depend on the wavelength range. In general the absorption difference between the two wavelength ranges becomes larger proportional to the difference between the central bandwidths of the two wavelength ranges used. The manufacturing tolerances causing spectral differences can be well controlled, but there is a need for different type of airway adapters, such as disposables for one time use and reusable for longer time use, which may comprise different property of optical windows with spectral differences. Thus the spectral tolerances within the group of airway adapter type may be small, but between the groups of different types of adapters the difference may be considerable.

The central wave length CWL and the band width BW of the first measurement wavelength range are adjusted to enable the absorption measurement of a certain gas at its specific wavelength. The more the measured gases, the more the measured wavelength ranges. Naturally in addition to gas specific absorption the measured wavelength comprises disturbing factors such as absorption caused by humidity, vapor, liquid or contaminated optical path. The CWL and the BW of the second wavelength range is adjusted to measure these disturbing factors, but to avoid the absorption of the measured gas. Thus the central wavelength of the first wavelength range is differing from the central wavelength of the second wavelength range. Also the band width of the first wavelength range may be different from the band width of the second wavelength range. In practice the CWL of the second wavelength range may be adjusted close to the CWL of the first wavelength range to measure similar disturbing factors to form a differential measurement, so that the disturbing factors can be eliminated from the first wavelength range to form a signal sensitive to measured gas only, but because of the physical nature of the optical BW the BWs of the first and the second wavelength ranges may overlap, which decreases the coefficient of efficiency. If the BWs of the first and the second wavelength ranges are separated from each other too much the measurement may become sensitive to disturbing factors.

The spectrum of the infrared radiation source may change during its life time as well as the spectrum of the receiving radiation sensing detector. Also the spectrum of optical path may change due to for example elongation, oxidation or mechanical shock.

The gas concentration signal is described by the signal difference between the first and second wavelength ranges. The offset error in one or between the two measured wavelength ranges generate a constant error to the gas concentration signal unless it is zeroed, where the zeroing means that the two wavelength range measurement signals are adjusted substantially equal in the reference gas, such as ambient gas atmosphere.

The measurement signal drift can be divided into two different types of drifts causing signal error. Offset drift, also called zero drift, is uniform signal error dependent on the signal level, whereas gain drift is altering signal error proportional to increasing or decreasing signal level. The offset drift can be zeroed, which means that the error is removed by readjusting the signal to its original signal level before the drift occurred. If for example the measured CO₂ concentration signal shows other than 0% value at 0% CO₂ ambient it can be zeroed to show 0% level of CO₂ concentration by removing the offset caused by the drift. The gain drift can be removed after the zeroing by calibrating the measurement at least at one gas concentration level, which is other than the zeroing level and readjusting the signal gain to correspond the target values. If for example the CO₂ concentration signal at 5% level of CO₂ shows a wrong value, even though the measurement has been zeroed and the offset drift has been removed already, the gain is readjusted to meet the 0% and the 5% values of CO₂ and to remove the gain error.

The removal of the offset error, in other words the zeroing, can be performed manually always when the user detects the need for that. Such situations can occur when the airway adapter is changed or when the gas analyzer shows other than the expected value, such as other than 0.04% of CO₂ when the analyzer is in the room air. Manual zeroing means that after detecting the error the user actively readjust the analyzer signal to meet the expected value. This is usually done through the host devices user interface software menus after the user has ensured that the zeroing can be performed for example changing a special zeroing cell including specific gas for that particular zeroing procedure. This is very complicated in usability sense and causes a risk of miss-calibrating the measurement, which can be a risk for the patient's health as the sensor shows wrong gas concentration values.

To ease and improve the gas analyzer usability and to reduce the miss-calibration risk the zeroing can be made to function e.g. semi-automatically or automatically to reduce the user's manual work. There are three different operating conditions that affect how and when it is reasonable to do the zeroing. The first condition is when the airway adapter 3 is removed from the coupling point 21 of the sensor 2, thus the sampling cell is not located between the optical path any more and the gas analyzer measures the ambient air. The second condition is when the airway adapter 3 is placed into the coupling point 21 of the sensor 3, but the airway adapter is not connected to the breathing circuit, thus sampling cell is located between the optical path, but the gas analyzer measures the reference gas e.g. from atmosphere like the ambient air or air fed into to the sampling chamber. The third condition is when the airway adapter 3 is placed into the coupling point 21 of the sensor 3 and the airway adapter is connected to the breathing circuit, thus sampling cell is located between the optical path and the gas analyzer measures the gas flowing in the breathing circuit. In any of the three conditions the absorption differences caused by the differences between airway adapter types can be corrected from the calculated gas concentration signal.

The detection of connecting and disconnecting the airway adapter to or from the coupling point of the sensor as well as detecting the airway adapter type can be implemented various ways, based on for example optical, electrical, electro-mechanical, magnetic or radio frequency. However, these detection methods need additional hardware into the sensor and/or the airway adapter making the construction more complex and more expensive. The more advantageous way to implement the place in detection of the airway adapter, without increasing the construction complexity or the cost, is based on measuring the at least two wavelength range signals, such as the first wavelength range signal and the second wavelength range signal. It also enables the detection of the airway adapter type, which information is needed after the zeroing, when the airway adapter is placed into the coupling point and the sampling chamber causes additional absorption, in addition to absorption of the measured gas, to adjust airway adapter specific correction coefficients so that the gas analyzer shows correct gas concentration values.

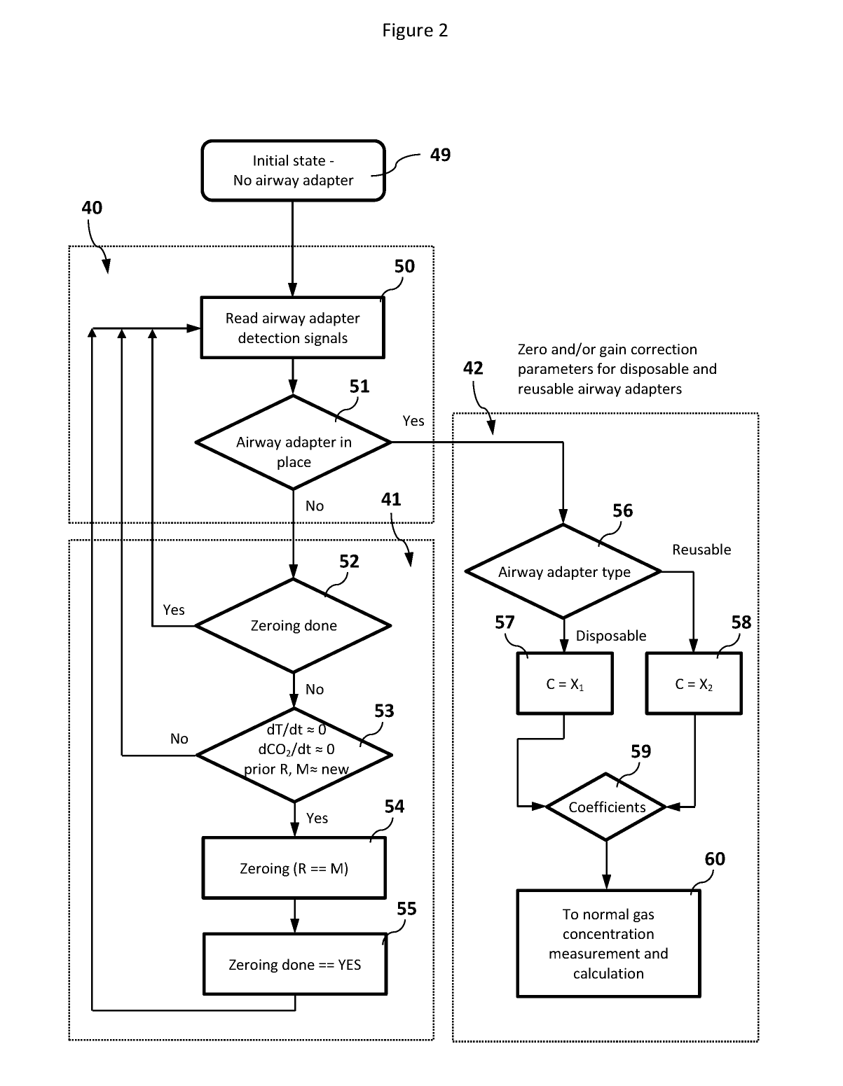

The offset error between the first and second wavelength range signals can be zeroed during all three described conditions, but the most advantageous way to perform zeroing may be when the airway adapter is removed from the sensor for the time of zeroing, as shown in the flow chart in FIG. 2. The zeroing is performed, when the airway adapter is detected as disconnected from the coupling point, by adjusting the first and second wavelength range signals equal or substantially equal, in which case the deviation converted into volume percent is advantageously less than 0.3 volume percent of the measured gas, more specifically less than 0.1 volume percent of the measured gas, or even more specifically less than 0.05 volume percent of the measured gas. After the zeroing when the airway adapter is detected as connected into the coupling point and when the airway adapter type is detected, with one of the detection methods described earlier, the airway adapter type specific compensation factors, depending on the airway adapter properties, are used to readjust the wavelength range signals equal or substantially equal.

When the airway adapter is not in place in the coupling point the amplitude of the first and second wavelength range signals are generally higher than that when the airway adapter is placed into the coupling point since the optical path of the sampling chamber is not absorbing the infrared radiation signal. When the airway adapter is placed into the coupling point of the sensor the amplitude of first and second wavelength range signals decrease rapidly to a lower level, whereas when the airway adapter is removed from the coupling point the amplitude of wavelength range signals return back to their original values rapidly. The amplitude differences of wavelength range signals between the different type of airway adapters, such as disposable and reusable airway adapters, depend on the differences between the optical path properties, such as optical window material and the optical path opening diameter. The optical window materials, which absorption is constant over the available spectrum as well as smaller diameter optical window opening decrease the signal amplitude at both wavelength ranges similarly, whereas materials having alternating infrared radiation absorption over the available spectrum decrease the amplitude of the two wavelength ranges unequally. It is also possible to have different optical designs for different airway adapter types on purpose to ease the separation of different airway adapter types from each other based on first and second wavelength range signal differences.

The zeroing, routine when the airway adapter is not placed into the coupling point of the sensor, is shown in a flow chart in FIG. 2 comprising various subroutines 40, 41 and 42. In an initial step 49, the sensor 1 is disconnected from the airway adapter 3. The routine may start from the subroutine 40 with reading the airway adapter detection signal(s) at step 50. If at step 51 the airway adapter is detected to be disconnected from the coupling point, i.e. it is not in place, which is a signal to the processing unit 25 that chances to enable the zeroing of gas analysis can be further studied in which case the processing unit can move to the subroutine 41. In case the airway adapter is in place the processing unit 25 instructs to move to the subroutine 42 as explained hereinafter. The subroutine 40 can be excluded if for instance a user can take care of it.

In case the processing unit 25 detects at step 52 that the zeroing was already done the normal gas component analysis can be done without need for zeroing again, but if it was detected by the processing unit or the user at step 52 that the zeroing was not already done at step 52, the chance to enable it can be determined at step 53. The zeroing is performed, if the sensor 2 is in an adequately stable state. The chance to enable the zeroing is based on the stability of the first and second wavelength range signals as discussed hereinbefore. The derivative of a gas signal such as dCO₂/dt and the absolute value of a gas concentration signal such as CO₂ derived from the wavelength range signals can be read. Also it is advantageous to determine whether a temperature of the at least one radiation sensing detector is substantially stable. This can be made in the processing unit 25 based on a temperature signal data provided by the temperature sensing element 13. If stable the derivative of the sensor temperature dT/dt is close to zero, in which case the dT/dt is advantageously less than 0.6 degrees of C [symbol] /min, more specifically less than 0.3 degrees of C [symbol] /min, or even more specifically less than 0.1 degrees of C [symbol] /min. Also the gas concentration signal and its derivative values are close to zero, such as CO₂and dCO₂/dt, indicate that the sensor is measuring stable, reference gas like ambient air gas concentration values, whereas the higher values indicate that the sensor is measuring gas concentration transients such as user's breathing gas interfering the zeroing as in this particular case with dCO₂/dt, in which case the processing unit determines that the signal data is not stable enough and may require restart the determination process again from step 50.

Thus the absolute value of the gas concentration and its derivative can be used to determine if the zeroing can be performed. To increase the zeroing quality the derivative of temperature can be used to find a thermally stable state for zeroing also. The values of dT/dt close to zero indicate that the sensor is in thermally stable whereas the high values indicate that it is experiencing high thermal transients that may disturb the sensor and fail the zeroing. Furthermore it is also advantageous to save the measured signal values that are used for zeroing into a memory 26 located into the sensor or the memory 27 located into the airway adapter to be used later on during the next zeroing. The stored values can be compared with the current values to find out high drift or component failures, but also to analyze the need for zeroing. If for example the gas concentration value, such as CO₂ shows its expected value and the new value is substantially the same as the previous within some limits, in which case the difference between the expected and the new value is advantageously less than 5%, more specifically less than 2%, or even more specifically less than 0.5%, there is no need for zeroing. However if there is a need for zeroing and all the signals used for analyzing that the sensor is in a sufficiently stable and in a correct state for zeroing the zeroing may be performed as shown more specifically with a subroutine 41, by adjusting the first and second wavelength range signals equal at step 54. After the zeroing, when the zeroing has been confirmed at step 55, the processing unit 25 waits until the airway adapter is connected into the coupling point 21 of the sensor 2 at step 51, as shown in the subroutine 40.

When the airway adapter is connected into the coupling point the airway adapter type is detected at step 56 and the gas concentration values corrected at steps 57, 58 and 59 with the airway adapter type specific factors, shown more specifically with a subroutine 42 in the FIG. 2. If the sensor is disposable the processing unit instructs to go through steps 57 and 59 to adjust airway adapter specific correction coefficients so that the gas analyzer shows correct gas concentration values with the disposable airway adapter. On the other hand if the airway adapter is reusable the processing unit instructs to go through steps 58 and 59 to adjust airway adapter specific correction coefficients so that the gas analyzer shows correct gas concentration values with the reusable airway adapter. After that at step 60 the processing unit 25 turns into a measurement mode until the airway adapter is disconnected from the coupling point again. The airway adapter type detection is based one of the methods explained hereinbefore. It is possible to have more steps, similar to steps 57 and 58, specific to airway adapter types that differ from each other.

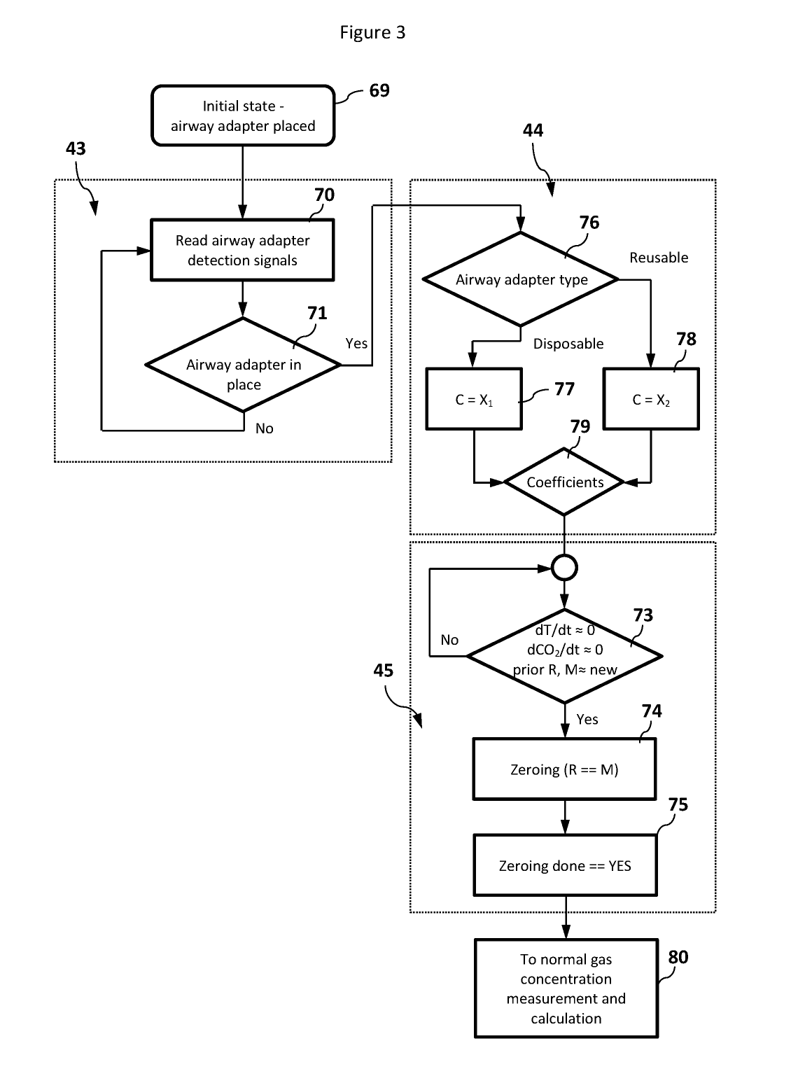

The zeroing can also be performed when the airway adapter is already connected into the coupling point and it may be connected into the breathing circuit as well or not. This zeroing routine is shown with the flow chart in FIG. 3. In an initial step 69, the sensor 1 is connected to the airway adapter 3. The routine may start from the subroutine 43 with reading the airway adapter detection signal(s) at step 70. If at step 71 the airway adapter is detected to be connected to the coupling point, i.e. it is in place, which is a signal to the processing unit 25 that chances to enable the zeroing of gas analysis can be further studied, in which case the processing unit can move to the subroutine 44. If the airway adapter is not connected to the coupling point of the sensor, the processing unit may return back and start from the beginning and continue from step 70 again.

If the airway adapter is connected to the sensor the airway adapter type is detected at step 76 and the correct airway adapter type specific coefficients are chosen at steps 77, 78, 79 to be used further in the zeroing and the gas concentration calculations, as shown with a subroutine 44 in FIG. 3. If the airway adapter is disposable the processing unit instructs to go through steps 77 and 79 to adjust airway adapter specific correction coefficients so that the gas analyzer shows correct gas concentration values with the disposable airway adapter. On the other hand if the airway adapter is reusable the processing unit instructs to go through steps 78 and 79 to adjust airway adapter specific correction coefficients so that the correct airway adapter type specific coefficients are chosen to be used further in the zeroing and the gas concentration calculations, as shown with a subroutine 45 in FIG. 3. It is possible to have more steps, similar to steps 57 and 58, specific to airway adapter types that differ from each other. The detection when the airway adapter is placed into the coupling point and the detection of the airway adapter type are based on similar techniques that explained hereinbefore.

The chance for enabling the zeroing, shown with a subroutine 45 in FIG. 3, can be determined after the step 79 by detecting whether the sensor is in an adequately stable state, which stability determination is based on reading the signals, such as the first and second wavelength range signals at step 73. This may include the absolute value and the derivative of a gas concentration signal, such as CO₂ and dCO₂/dt derived from the first and second wavelength range signals. Also as explained hereinbefore the derivative of the sensor temperature dT/dt at step 73 can be exploited when determining the chance to enable the zeroing.

The first and second wavelength range signals, gas concentration signal and its derivative values, such as CO₂ and dCO₂/dt, close to zero indicate that the sensor is measuring stable, reference gas, such as ambient air, concentration values, enabling zeroing at step 74. The performed zeroing may be confirmed at step 75. Instead the higher values indicate that the sensor is measuring gas concentration transients such as the user's or the patient's breathing gas interfering the zeroing, in which case the processing unit may instruct to go back and start again from step 73. If the airway adapter is connected to the breathing circuit, which is further connected to patient and the ventilator, the sensor should measure the patient's breathing cycle including inspired and expired gas concentrations at step 80.

Thus the absolute value of the gas concentration together with its derivative can be used to determine if the zeroing can be performed, which is when the gas concentration is close to its reference value used for zeroing and its derivative close to zero indicating that the gas concentration is not changing. This would be when there is a constant flow of reference gas used for zeroing, such as ambient air, through the airway adapter. This may be even during the inspiration or more specifically the end of the inspiration when the possible dead volume in the breathing circuit has been flushed with the reference gas so that the absolute gas concentration values are close to the reference gas values and the change of gas concentration values (dGasconcentration/dt) are close to zero.

To increase to zeroing quality the derivative of temperature can be used to find a thermally stable state for the zeroing also. The values of dT/dt close to zero indicate that the sensor is in thermally stable whereas the high values indicate that it is experiencing high thermal transients that may disturb the sensor and fail the zeroing. Also the breathing gas pressure information from the pressure sensor measuring the pressure inside the breathing circuit, which sensor may be located for example inside the ventilator, the airway adapter of similar place, can be used to correct the gas concentration error caused by the pressure. Furthermore it is also advantageous to save the measured signal values that are used for zeroing into a memory 26 located into the sensor or the memory 27 located into the airway adapter to be used later on during the next zeroing. The stored values can be compared with the current values to find out high drift or component failures, but also to analyze the need for zeroing. If for example the gas concentration value, such as CO₂ shows its expected value and the new value is the same as the previous within some limits there is no need for zeroing. However if there is a need for zeroing and all the signals used for analyzing that the sensor is in a sufficiently stable and in a correct state for zeroing the zeroing is performed, shown more specifically with a subroutine 45, by adjusting the wavelength range signals equal or substantially equal. After the zeroing the sensor turns into a normal measurement mode at step 80 until the airway adapter is disconnected from the coupling point again. If the sensor is not stable enough to be zeroed the subroutine 45 returns back to its beginning. The airway adapter type detection is based one of the methods explained earlier.

The embodiments discussed hereinbefore solve many problems relating to signal errors of conventional mainstream measurement of gases. In the anesthesia and transportation the need for monitoring the same subject is normally hours and airway adapters are changed between the patients many times per day to avoid cross contamination. In intensive care the subject is usually monitored much longer time up to months, but during that period of time the airway adapter is often replaced several times or at least once every 1-3 days, as they get contaminated of mucus, bacteria, viruses etc. The replacement of airway adapters as well as drift and signal changes caused by the other components and the optical path of the analyzer causes need for zeroing the gas analyzing measurement possibly many times per day. Disregarding the zeroing leads into measurement signal error that may cause risk for the patient's health. Also the manual zeroing that needs lot of users work and attention may lead into zeroing errors that may cause risk for the patient's health, but the zeroing also reduces the time needed for taking care of the patient. The zeroing procedure comprising detection when the airway adapter is placed into the sensor, the detection of the airway adapter type and the detection and the procedure for zeroing the signal errors disturbing the gas analyzes according to this embodiment solve these above mentioned problems, releasing the care giver to concentrate on the patient.

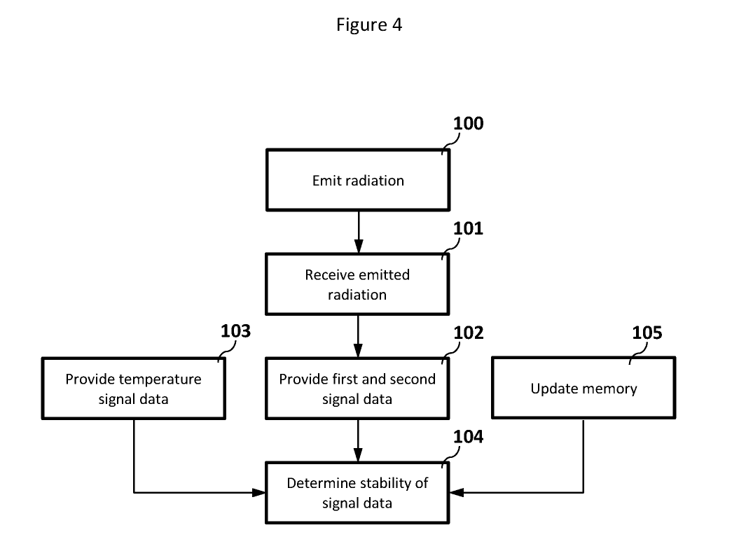

As can be understood step 53 as well as step 73 includes various further steps to determine the chance to enable the zeroing of the gas analysis. In FIG. 4 at step 100 the at least one radiation source emits radiation, such as infrared radiation, which is received at step 101 by the at least one radiation sensing detector. This radiation received by the detector comprises a first wavelength range which may be absorbed by the at least one desired gas component of the respiratory gas in case such gas component is present to the extent exceeding normal reference gas concentration, such as atmospheric gas concentration. The desired gas component may be e.g. carbon dioxide. The first wavelength range may also be absorbed by one or more disturbing factor explained hereinbefore, in case such factor is present. Also the radiation received by the detector comprises a second wavelength range which may be absorbed or even be absorbed substantially only by the disturbing factor. These first and second wavelength ranges may have central wavelengths differing from each other. Also the first wavelength range typically differs from the second wavelength range.

The at least one radiation sensing detector provides at step 102 the processing unit a first signal data indicative of an amount of the at least one desired gas component and indicative of the disturbing factor, and a second signal data indicative of the disturbing factor. Also the stability of the temperature signal data is provided from the temperature sensing element as an option to the processing unit at step 103.

The processing unit may determine at step 104 a stability of the first and second signal data as a function of time, and if they are substantially stable the zeroing is enabled at step 54 or 74 shown in FIGS. 2 and 3 to improve the measurement accuracy. In case the temperature signal data is available the processing unit may exploit at step 104 it when determining a chance to enable the zeroing. If the temperature signal data is stable enough it supports to enable the zeroing at step 54 or 74. In normal operation outside the zeroing, i.e. when the zeroing is not needed or is difficult to do, the gas analysis unit can be used to gas analysis by obtaining the signal which is indicative of at least one desired gas component of a respiratory gas.

As shown in FIG. 4 the first and second signal data from the at least one radiation sensing detector may be updated at step 105 as an option into the memory 26, 28 periodically by the processing unit. The processing unit may compare the last received stable signal data to the corresponding signal data in the memory received earlier. If they are substantially stable the zeroing is enabled again.

The written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for determining when to enable a zeroing of gas analysis during ventilation of a patient, said gas analysis unit being configured outside the zeroing to obtain a signal indicative of at least one desired gas component of a respiratory gas for an analysis, said method comprising:
emitting radiation by means of at least one radiation source;
receiving emitted radiation in at least one radiation sensing detector, said received radiation comprising a first wavelength range configured to be absorbed by said at least one desired gas component of the respiratory gas in case such gas component is present to the extent exceeding a reference gas concentration, which first wavelength range also being absorbed by one or more disturbing factor, in case such factor is present, and a second wavelength range configured to be absorbed by said disturbing factor but not to be absorbed by said at least one desired gas component of the respiratory gas, said first wavelength range differing from said second wavelength range;
providing by means of said at least one radiation sensing detector to a processing unit a first signal data indicative of a concentration of said at least one desired gas component and indicative of absorption of said disturbing factor, and a second signal data indicative of absorption of said disturbing factor; and
determining in said processing unit a stability of said first and second signal data as a function of time, and if they are substantially stable enabling the zeroing to improve a measurement accuracy.

2. The method according to claim 1, further comprising providing in a temperature sensing element temperature signal data of said radiation sensing detector to said processing unit to determine whether the temperature is substantially stable, and if said first signal data, said second signal data and said temperature signal data are substantially stable said processing unit is configured to enable the zeroing.

3. The method according to claim 1, further comprising updating by means of said processing unit into a memory periodically said first and second signal data from said at least one radiation sensing detector and if they are substantially stable enabling the zeroing again in said processing unit.

4. The method according to claim 3, wherein said determining comprises comparing in said processing unit last received stable first and second signal data to corresponding earlier received signal data in said memory.

5. The method according to claim 1, wherein said emitting radiation is configured to permit the radiation to pass through an airway adapter to said at least one radiation sensing detector, said airway adapter being filled instead of the respiratory gas with a reference gas for the zeroing.

6. The method according to claim 1, wherein said emitting radiation is configured to permit the radiation to bypass an airway adapter through a reference gas before enabling the zeroing, which airway adapter is configured outside the zeroing to receive the respiratory gas flow for the analysis.

7. The method according to claim 1, further comprising detecting in said processing unit whether or not an airway adapter is in place, which airway adapter is configured to receive either a reference gas or the respiratory gas.

8. The method according to claim 7, further comprising recognizing in said processing unit a type of said airway adapter based on a change in amplitude of said first and second signal data.

9. The method according to claim 1, wherein said zeroing comprises substantially equalizing said first signal data and said second signal data.

10. The method according to claim 1, wherein said first and second wavelength ranges comprise central wavelengths differing from each other.

11-20. (canceled)
